(19) United States
(12) Patent Application Publication
(10) Pub. No.: US 2019/0259495 A1
(43) Pub. Date: Aug. 22, 2019

(54) Direct Data Transfer Devices to Hand Off Patient Responsibility

(71) Applicant: Vladimir Fridman, Brooklyn, NY (US)

(72) Inventor: Vladimir Fridman, Brooklyn, NY (US)

(21) Appl. No.: 15/898,252

(22) Filed: Feb. 16, 2018

(51) Int. Cl. G16H 40/20; G16H 40/63; H04W 4/80; H04W 4/029

(52) U.S. Cl. CPC G16H 40/20; H04W 4/029; H04W 4/80; G16H 40/63

(57) ABSTRACT

Wireless transceivers of a plurality of health care professionals or other caregivers are used in embodiments of the disclosed technology. Through direct communication between the transceivers, a plurality of patients are associated with each wireless transceiver. While the patients under the care of each professional change over time, in order for one professional to end his or her responsibility of patients, a direct hand-off of data between his or her devices and that of another caregiver must take place. The other party associated with the direct hand-off than assume responsibility and this is recorded and can be retrieved by others. This ensures that the current responsible caregiver can be located at any given time and that a responsible caregiver is available for each patient and can be contacted at any given time.

### BACKGROUND OF THE DISCLOSED TECHNOLOGY

A frequent problem is health care facilities such as hospitals is knowing who is responsible for which patients. Take the example of a typical floor on a hospital with a nursing station in the middle surrounded by one or multiple dozens of patient rooms. Doctors, nurses, cleaning staff and other caregivers may be responsible for the care of some or all of the patients on the floor and other floors. Each caregiver may be shuffling between rooms, answering calls, making rounds, and so forth. Each is busy with their own job. However, caregivers often need to call on the aid of others. For example, a nurse may need to a summon a physician only to find the physician on call has left for the day. The hospital typically isn't left without a physician on the premises, but then the nurse must figure out who the physician is that has taken over the duties of the physician who left or is otherwise occupied with another task and not currently responsible for the particular patient.

Delays in patient care are, at best, costly. Extra time is needed just to find the professional who can care for the patient rather than actually providing the care. Further, such delays keep hospital beds occupied longer an unavailable for others while it sometimes takes hours, for example, just to have a doctor sign a discharge order. Meanwhile, the room where the patient is residing cannot be cleaned and prepared for the next patient and backlogs develop. At worst, delays in care result in a worsening of a medical condition which should have and could have been treated sooner had the hospital and staff been able to be more organized and communicative.

This is an undesirable situation which plays itself out in hospitals and other places of patient care day after day throughout the United States and perhaps the world. The problem that must be solved is how to ensure that patient receive proper and timely care and caregivers spend more time giving care rather than having to find those who can. This would increase the overall patient experience as well as medical treatment while decreasing overhead for facilities such as hospitals.

These and other limitations of the prior art are solved by embodiments described in the present disclosure.

### SUMMARY OF THE DISCLOSED TECHNOLOGY

Embodiments of the disclosed technology utilize a short range communication system and method based on distribution of at least a first and second short range communication device with a data storage device to a first and a second health care practitioner. Such practitioners can include doctors, nurses, and other caregivers who are each assigned to take care of or be a temporary guardian (person of responsibility) for another person. In order to keep track of whom each practitioner is responsible for, a unique identifier stored in each of the data storage devices of each short range communication device is associated with a respective health care practitioner. This is, for example, a serial number programmed in read only memory which is then associated with a respective health care practitioner by way of this practitioners biometric data (e.g. fingerprint or iris scan) or a user name and password to log in to a system and make edits to the associations between a caregiver and patient. Further, electronic exchanges of data with a short range communication device of a patient or one under the care of the caregiver can be used to associate the patient (herein, defined as any person under the medical care or temporary guardianship of another) with the particular caregiver. In this manner, a first plurality of patients are associated with a first health care practitioner.

Then an indication is received that a particular patient of the plurality of patients is no longer under care of the first health care practitioner and is thus removed from being associated with the first practitioner. This can be by another caregiver or worker indicating that the patient has been discharged, the patient voluntarily exiting from being under care, or the patient moving to the care of another. As such, the association between the first caregiver and patient is removed.

An indication received by a network node that a wireless electric transmission of data between the short range communication device of the first health care practitioner with a short range communication device of the second health care practitioner. This communication which entails a direct data exchange between the devices of the first and second practitioner indicates that all patients associated with the first health care practitioner are now associated with the second. The direct data exchange is, for example, the result of one health care practitioner ending his shift while another takes over the patients (previously) under the responsibility of the first health care practitioner, at the start of a new shift. The direct data exchange might be an outgoing practitioner holding his communication device near that of an incoming health care practitioner, causing an electronic handshake and trading of data, which updates a system storing associations of each patient with a caregiver.

The direct data exchange can be a one way or two way exchange of data. In a one way exchange of data, for example, the second practitioner receives a transmitted code associated with the first practitioner and and the data is sent to a central server which stores and/or propagates present patient associations to those who seek this information. Information about each of the first plurality of patients can received directly to a handheld wireless transceiver associated with said second health care practitioner after the said step of receiving the transmission of data. In this manner, the practitioner taking over care can directly receive information about those who he/she is caring for, and then can distribute (such as via wireless transmission) this information to the central server. The patient information can include patient care data (defined as a description of care given to the patient previously and/or ongoing requirements to care for the patient) and/or patient vital statistics. Such patient care data and/or the patient vital statistics are unavailable, in some embodiments of the disclosed technology, to the second health care practitioner until after the step of receiving the indication of the wireless electric transmission of data. The communication devices used for the direct data transmission can be wrist bands and/or can also communicate with a network node to a larger network. Data from (communicated via) this network node is made available to at least a separate device with network connectivity confirmed to be operated by a third health care professional.

Described another way, a short range communication device is distributed to a plurality of health care professionals. Based on an unique identifier within each of the short range communication devices, each short range communication device thereof is associated with one of a plurality of health care professionals. Via a network node, an indication of at least one patient under care of a first health care professional of the plurality of health care professionals is received. An indication that a direct data exchange has taken place between two of the short range communication devices is then received, wherein a first of the short range communication devices is associated with the first health care professional and a second of The short range communication devices is associated with a second of the health care professionals. Then via a network node, a request for care of the at least one patient is received and based on the direct data exchange, the second health care professional is contacted for care of the patient.

Upon detecting that the first health care professional has exited from premises (a circumscribed location of patient care such as the walls of a building), a reminder via a packet-switched network node is sent to a handheld device previously determined to be operated by the first health care professional to transfer the patient to another of the health care professionals. In some embodiments, on determining that a work shift is over for the first health care professional, a reminder is sent via a packet-switched network node to a handheld device previously determined to be operated by the first health care professional to transfer the at least one patient to another of the health care professionals.

The short range communication devices can communicate patient identifying information to one another, representative of patients whom are under care of The first health care professional. after communication of the patient identifying information, at least a device previously determined to be operated by the second health care professional is sent a notification that the second health care provider is responsible for the at least one patient. A query, in embodiments of the disclosed technology, is received via a network node requesting contact information for a responsible health care professional for the at least one patient and a response is returned indicating a latest recipient of the direct data exchange between the two said short range communication devices. At least one of the short range communication devices communicates information with patient identifying information to a device providing data sufficient to answer the query in some embodiments of the disclosed technology. In other embodiments, the short range communication devices communicate substantially only the unique identifier to each other, and substantially only the unique identifier is transmitted wirelessly to a data storage device which stores an association between the at least one patient and, at any given time, one of the plurality of health care professionals.

A fixed position reader (defined as “a device which receives data with a unique identifier of a short range communication device associated with a health care professional, the device being fixed to a wall or other immovable object”; for purposes of this disclosure, “short range” is defined as “defined to function between zero and one meter away” and in some embodiments, is less than 15 centimeters away) with a transceiver communicates, in one embodiment of the disclosed technology, with short range communication devices. The step of receiving an indication that the direct data exchange has taken place further includes steps, in this embodiment, of having the fixed position reader receive data from said short range communication device associated with said first health care professional sufficient to indicate that the at least one patient associated with the first health care professional is now associated with the second health care professional. The first position reader also, i this embodiment, can receive data from the short range communication device associated with the second health care professional sufficient to indicate that the at least one patient associated with the first health care professional is now associated with the second health care professional.

A method of patient care can also include carrying an identification device with wireless connectivity, locating or being located by another health care professional whose duties are to be assumed, placing the identification device close enough and for a long enough time period until a wireless data exchange takes place between the identification device and that of a second identification device associated with the professional whose duties are to be assumed, causing information about the wireless data exchange to be transmitted via a network node to a server, and receiving from the server an indication of patients of the another health care professional for which duties are assumed.

Any device or step to a method described in this disclosure can comprise, or consist of, that which it is a part of, or the parts which make up the device or step. The term “and/or” is inclusive of the items which it joins linguistically, and each item by itself. Any object described can be as described or “substantially” as such wherein “substantially” is defined as “at least 95% true” or “at least 95% of the amount specified.”

### BRIEF DESCRIPTION OF THE DRAWINGS

FIG. 1 is a high-level block diagram of devices used to carry out embodiments of the disclosed technology.

FIG. 2 shows an example of a RFID transmitter or receiver worn as a wrist band and a mounted transmitter or receiver used in embodiments of the disclosed technology.

FIG. 3 is a flow chart showing a method of carrying out embodiments of the disclosed technology.

FIG. 4 is a high-level bock diagram of devices used to carry out embodiments of the disclosed technology.

### DETAILED DESCRIPTION OF EMBODIMENTS OF THE DISCLOSED TECHNOLOGY

Wireless transceivers of a plurality of health care professionals or other caregivers are used in embodiments of the disclosed technology. Through direct communication between the transceivers, a plurality of patients are associated with each wireless transceiver. While the patients under the care of each professional change over time, in order for one professional to end his or her responsibility of patients, a direct hand-off of data between his or her devices and that of another caregiver must take place. The other party associated with the direct hand-off than assume responsibility and this is recorded and can be retrieved by others. This ensures that the current responsible caregiver can be located at any given time and that a responsible caregiver is available for each patient and can be contacted at any given time.

Embodiments of the disclosed technology are described below, with reference to the figures provided.

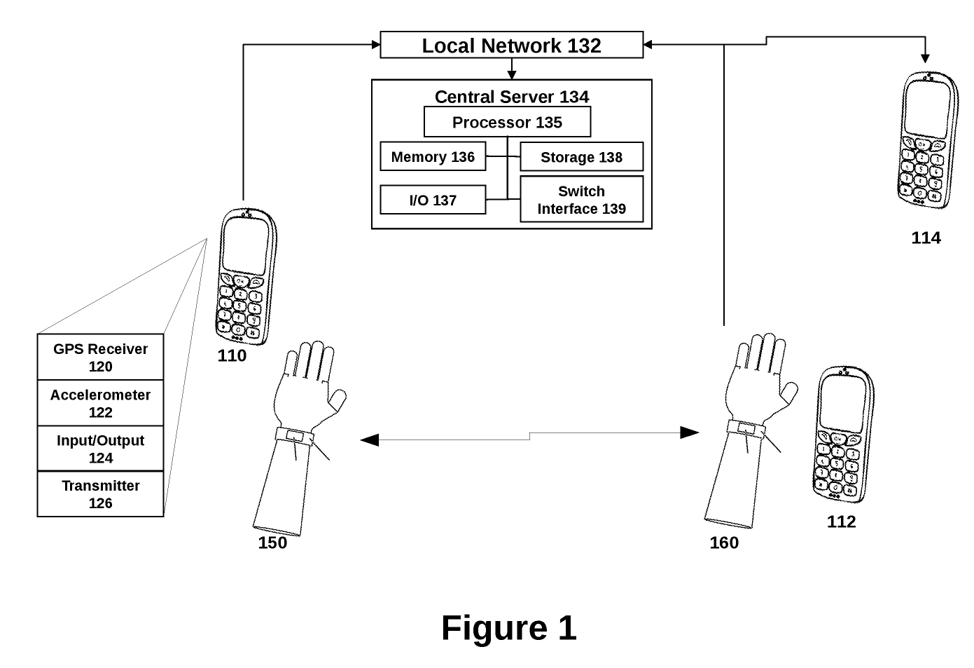

FIG. 1 is a high-level block diagram of devices used to carry out embodiments of the disclosed technology. A first hardware device 110 has a network connection to a packet switched network (e.g. the global network of packet-switched routers, hubs, switches, and nodes used to transport data to each other by the TCP/IP protocol known as “the Internet”) or a local network 132 (a private network behind a specific router or set of routers or hubs which can be accessed only by others who are connected within the specific network or via a virtual private network). Through the packet switched network 132, in this example, various hand-held wireless transmission devices, such as devices 110, 112, and 114, can communicate. A transmitter/receiver can be used to enable wireless transmission and receipt of data via the packet-switched network 130, such as by way of the 802.11 wireless transmission protocols known in the art. Alternatively, a wired connection such as via category 5 or 6 cable can be used.

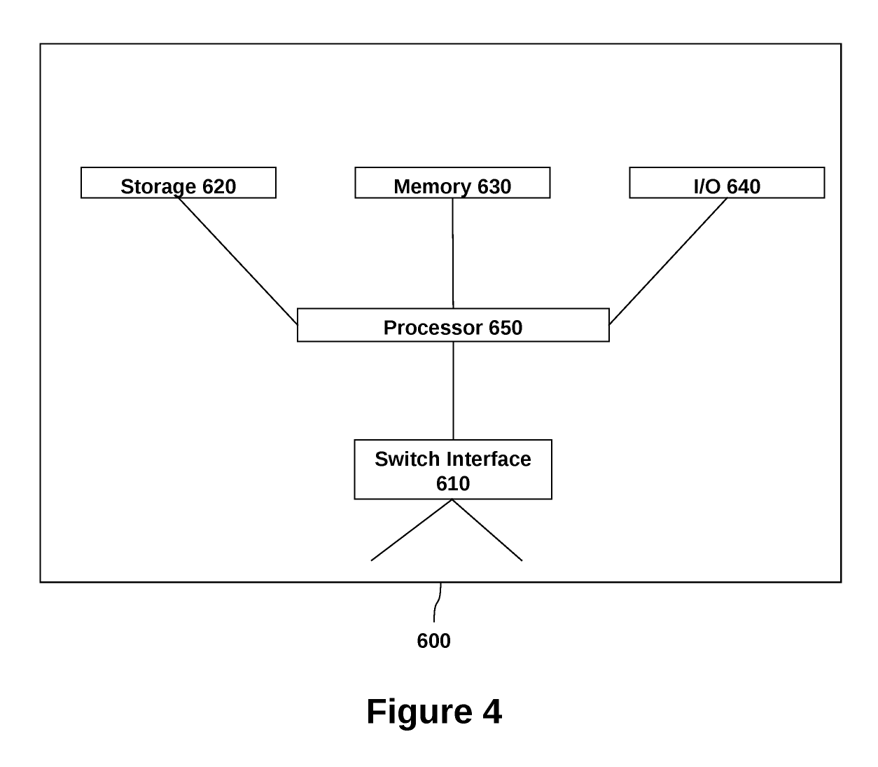

This network, in embodiments, interfaces with a central server 134 and/or a server (another hardware device or multiple different hardware devices, such as described with reference to FIG. 4). The central server receives communications from the packeted switched network/local network 132. Versions of data, which include portions thereof, can be transmitted between the devices. A “version” of data is that which has some of the identifying or salient information as understood by a device receiving the information.

Referring again to the local network 132, this network can have a switch which interfaces with a telephone network including a GSM network, SMS network, or another network or protocol defined for use with phones and/or phone service. Such phone and/or phone service is a distinctly different network than the packet switched network 130, though data from one network can and sometimes is carried via the other network (e.g. a TCP/IP connection by way of an analog modem or a phone connection carried via a packet switched network). For purposes of this disclosure, in some embodiments of the disclosed technology, at least the protocol used to communicate between the hardware devices and the telecom switch is a different protocol than the one between the hardware devices 110, 112, and 14 and the packet switched network 132. In some embodiments, not only is the protocol different but so is at least some or all of the network nodes and hardware switches that the data is transported over between the hardware device and respective network.

Each device shown in FIG. 1 represents a device and node where data are received and transmitted to another device via electronic or wireless transmission, Each can be connected to, or communicate via, a central server 134 which forms a hub, such as operated by an entity controlling the methods of use of the technology disclosed herein. This hub has a processor 135 which processes data sent and received to the end user hardware devices 110, 112 and 114 and determines when security credentials have been met to grant a hand-off of association of a patient to another caregiver. Definitions for these terms are provided in the “Summary” above. This hub 134 further has memory 136 (volatile or non-volatile) for temporary storage of data, storage 138 for permanent storage of data, and input/output 137 (like the input/output 124), and an interface 139 for connecting via electrical connection to other devices.

Discussing the handheld wireless transceivers themselves, they can have within multiple transmitters 126 such as a Bluetooth transceiver and a WiFi transceiver where thew Wifi transceiver communications to the local network 132 and central server 134 and the Bluetooth transceiver is of shorter range and communicates with a wearable identification device 150 or 160, for example. The shorter range transmitter 126 can have a transmission range of 20 meters, 10 meters, 5 meters, 1 meter, or even a fraction of a meter such as 10 centimeters or less (near field communication). In this manner, transmission between the identification device 150 and the wireless transceiver 110 (or the like) takes place only when in very close proximity. Still further, in embodiments of the disclosed technology, identification devices 150 and 160 communicate directly with each other to indicate a hand-off of patients from one caregiver to another. Such a transmission can instead be from one caregiver associated device (110 or 150) to that of the other caregiver (160 or 112).

The wireless transceivers can further have a GPS (global positioning system) receiver 120 and/or an accelerometer 122 to determine velocity and location. If a location is far away from a location of care (for example, out of a pre-circumscribed and defined geographic location with metes and bounds entered into, for example, the central server 134) a hand-off of patients can be considered invalid and ignored. So too, if the hand-off occurs at a non-zero velocity, then it can be determined to be erroneous unless, in one embodiment, this occurs while rising or falling only within a specific range bound velocity or acceleration such as to allow a hand-off in an elevator. The input/output 124 allows a caregiver to interact with the device such as via a touchscreen and display.

The additional device 114 shown can be associated with (defined as, in relevant embodiments of the disclosed technology, “authenticated as being under the operation of”) of a non-caregiver while the devices 110 and/or 112 are determined to be associated with that of a first and second caregiver in embodiments of the disclosed technology. The operator of the device 114 might, for example, request to find which doctor is caring for a certain patient, and based on the data hand-off between devices 150→112, 150→160, 110→160, and/or 110→112, it is determined which physician is currently caring for the certain patient. Such a result is then given to the operator of device 114 by way of the device itself, or via the device 114, the operator thereof is put in contact with the responsible physician (or caregiver). Thus, in an embodiment of the disclosed technology, when an operator of device 114 looks up a specific patient and then requests to contact a caregiver for the patient, the device 114 calls or opens a communication channel to the device 112, for example, if the operator of device 112 is the present caregiver of the specific patient as determined by a hand-off of patient responsibility using, for example, a direct data exchange between devices 150 and 160. It should further be understood that the device 114, though shown as a handheld wireless device can be any computational device including a fixed in place and wired computer device.

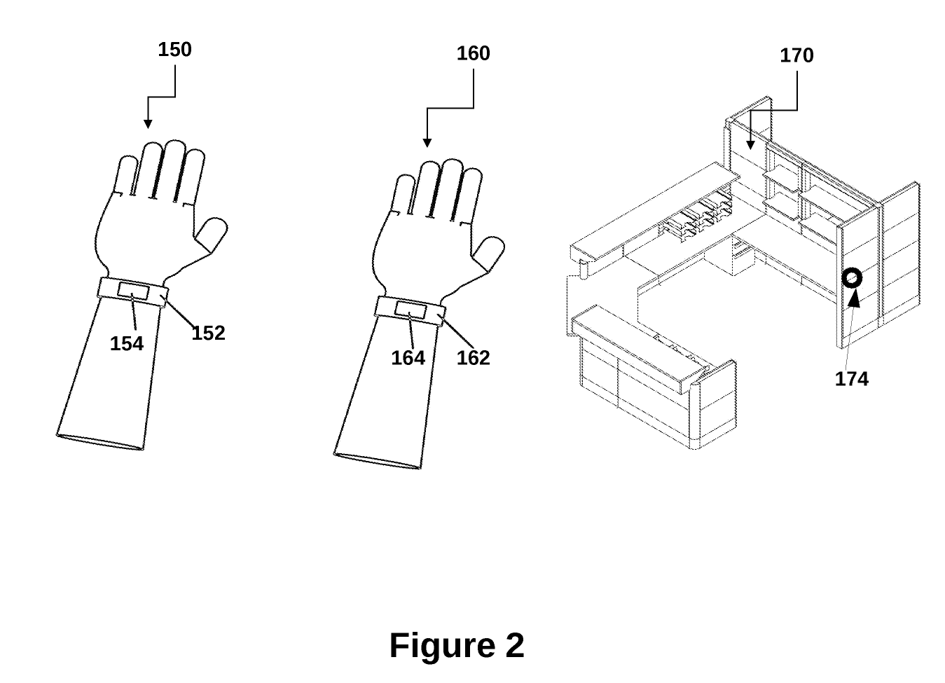

FIG. 2 shows an example of a RFID transmitter or receiver worn as a wrist band and a mounted transmitter or receiver used in embodiments of the disclosed technology. It should be understood that in embodiments of the disclosed technology, what is described with reference to the devices 150 and 160 can be in a card (such as an identification badge), integrated into a wireless hand-held transceiver (such as device 110 and/or 112), or otherwise. The method of transmission or receipt of data can be any known in the art, such as a wireless transmission, bar code read, or the like, though RFID is shown in FIG. 2. RFID is radio frequency identification and in embodiments thereof requires low power or no power. The circuitry is powered by propagating radio waves and a transmission is then made, such as of a unique identifier stored on a storage device of an RFID transceiver 164 held on a wrist-band 162 or the like.

Alternatively, the transceiver 164 can be a Bluetooth or other short range or near field communication device which communicates by syncing with another device so that, for example, transceivers 154 and 164 transmit data to each other while held on, and associated with, a respective caregiver 152 and 162. In this manner, patients associated with a first caregiver, such as a caregiver wearing a bracelet 152 are then associated with a second caregiver, such as one wearing the bracelet 162.

A standalone or fixed position station 170 (one which is designed to remain in place for a period of at least 30 days without any movement, of movement of more than 50 cm relative to the ground) can also be used to receive a patient assignment or discharge from an assignment of patients. In this manner, a fixed position receiver 174 (fixed position being defined as above with reference to the station) is fixed to the fixed position station 170, a wall, or a desk, or the like and receives from, or communications with, a device 150 or 160. This can be done at the beginning or end of a work shift, a break, or the like. In embodiments of the disclosed technology, a caregiver/professional receives their patient assignments/associations by way of using the fixed position receiver 174 and/or discharges such assignments/associations in this manner.

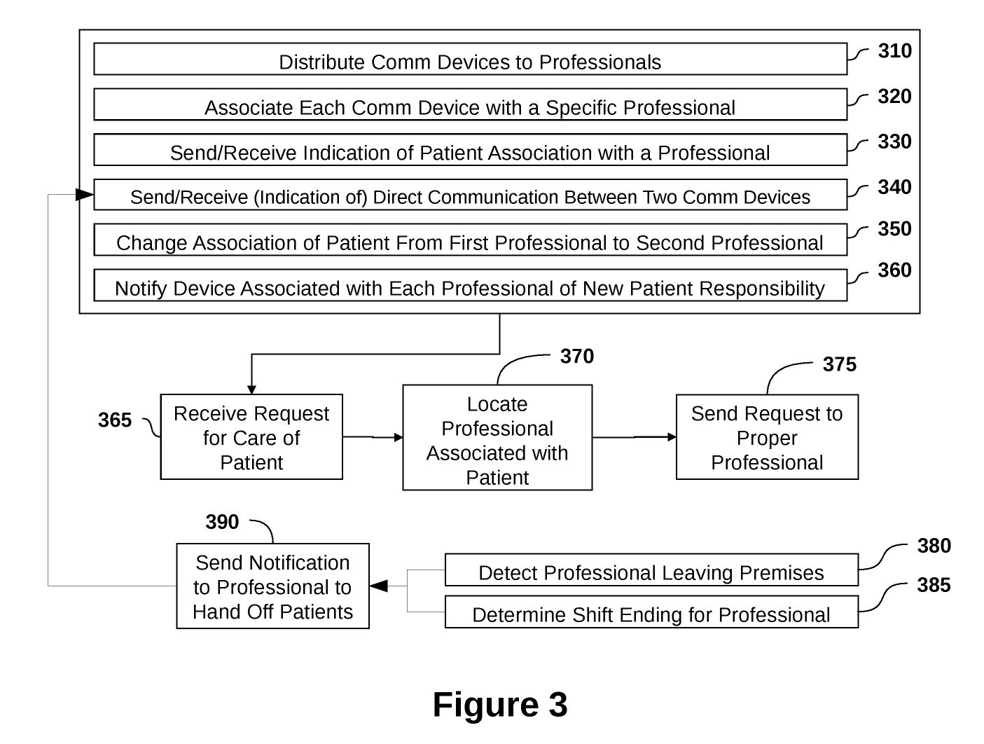

FIG. 3 is a flow chart showing a method of carrying out embodiments of the disclosed technology. In step 310, communication devices such as those shown in FIG. 2 are distributed to a plurality of health care professionals (also referred to as “professionals” or “caregivers” in this disclosure). Each communication device is associated with a specific professional in step 320. This may be, for example, by recording associating one of the communication devices with a wireless handheld transceiver authenticated for use by one of the caregivers or by recording a unique identifier of one of the communication devices in a database entry with a specific caregiver. Once a communication device, such as a wrist band with RFID (see FIG. 2) or the like is associated with a specific caregiver, then patients are associated with the caregiver in step 330. This step can take place prior to steps 310 and 320 whereby patients are associated with a specific caregiver meaning that the patients are under the care and/or responsibility of the caregiver.

At this stage, there are now communication devices each associated with a specific caregiver as well as patients associated with as specific caregiver. The association between patients and caregivers can change in step 350 (which can be carried out before, after, or at the same time as any of the other steps shown in FIG. 3). This can happen, for example, when a new patient enters a hospital and is assigned a doctor on call or a patient is moved from one section of a hospital to another and is now under the care of another physician. This can also happen with, for example, infants checked into daycare and a daycare worker beginning a shift or leaving for a break. Further, step 350 can be carried out based on step 340 where direct communication takes place between two communication devices, each associated with a specific professional. In this manner, patient responsibility is handed off from one professional to another for all patients associated with one of the professionals.

In step 340, the direction communication between two communication devices can take place in any of the following ways. Two professionals, each with a communication device (bracelet, name badge, or the like) place their devices in close proximity when handing off patients. One “sends” while the other “receives”. A button, switch, or simply the context may determine who is the sender and who is the receiver. A professional with no associated patients at present and/or whom more recently entered a health care location or activated his communication device may be designated as the receiver. Conversely, the professional with more associated patients and/or for a longer period of time may be the sender. One can also move a switch from “send” to “receive” position which might simply disable/enable RFID circuitry on one's device and/or activate battery power for a Bluetooth receiver which then receives the unique identifier from the other device and transmits same via a short range wireless device to a handheld wireless transceiver. Depending on the embodiment, this can be carried out in one of two directions or bidirectionally between a first professional and associated devices and a second professional and associated devices. That is, step 340, the direct communication between the devices can be a transmission of a patient identifying data or simply a unique identifier of the first professional to a communication device associated with a second professional or vice versa.

In step 360, each professional is notified of their new patient responsibility. Thus, supposing the first professional was responsible for patients Ackerman, Braverman, Cohn, and Davenport. After the direct communication between, for example, their wristbands (e.g. devices 150 and 160 of FIG. 1), responsibility for care of Ackerman, Braverman, Cohn, and Davenport is passed to the second professional. The wristband itself may now list “empty” or “no patients” to the first health care professional while that of the second lists the patients Ackerman, Braverman, Cohn, and Davenport. Similarly, in other embodiments this data may be displayed on a handheld wireless transceiver associated with each respective professional/caregiver (e.g. devices 110 and 112 of FIG. 1).

Referring now to steps 365 through 375 of FIG. 3, suppose someone wishes to find who is responsible for a particular patient at a particular time. A nurse, for example, may need to call on the attending or responsible physician to provide immediate medical attention, change a dosage of medication, discharge a patient, or the like. Using, for example, device 114, a handheld or standalone communication device which can access data stored on the central server 134 of FIG. 1, a third party may want to request care for a particular patient in step 365. Based on some or all of steps 310 through 360, this particular patient is associated with a particular professional and this association might be stored in the storage device 138 of the central server 134. In step 370, this data is retrieved as the professional associated with the patient is located and reported to the third party requesting same. The information request is also sent, in some embodiments of the disclosed technology, in step 375, to a handheld wireless transceiver associated with the professional whom is currently caring for the patient. Thus, a request for assistance is made using the patient as the identifier but the request is made to the attending physician/professional, in some cases, without the requester having knowledge of whom the responsible caregiver is before, during, and/or after care is given.

Now referring the bottom row of FIG. 3, steps 380 through 390, in step 380 it is detected that a professional is leaving the premises. This can be determined in a variety of ways such as the handheld wireless transceiver (e.g. device 110) becoming disconnected from an 802.11 network for a pre-defined period of time, the GPS receiver 120 in the professionals device reporting a location outside of a geo-fenced (“geographically fenced”—a pre-determined geographical boundary) of the location where care is administered, and/or an acceleration or velocity of the handheld wireless transceiver above a certain threshold indicating that the professional is likely driving rather than walking (e.g. traveling at a speed higher than that which would occur when care is being given at a particular location). In addition or alternatively, step 385 can be carried out where it is determined that the work time for a particular professional is coming to an end or has ended. The professional, via his/her handheld wireless transceiver is then sent, in step 390, a notification to hand off patients to another. When the professional does so, step 340 is carried out (again) and there is direct communication between a transmitter of one device and a receiver of another, each device associated with a specific professional. In this manner, it is ensured that the current responsible caregiver can be located at any given time and that a responsible caregiver is substantially always available for each patient and can be contacted at any given time.

FIG. 4 is a high-level bock diagram of devices used to carry out embodiments of the disclosed technology. Device 600 comprises a processor 650 that controls the overall operation of the computer by executing the device's program instructions which define such operation. The device's program instructions may be stored in a storage device 620 (e.g., magnetic disk, database) and loaded into memory 630, when execution of the console's program instructions is desired. Thus, the device's operation will be defined by the device's program instructions stored in memory 630 and/or storage 620, and the console will be controlled by processor 650 executing the console's program instructions. A device 600 also includes one, or a plurality of, input network interfaces for communicating with other devices via a network (e.g., the Internet). The device 600 further includes an electrical input interface. A device 600 also includes one or more output network interfaces 610 for communicating with other devices. Device 600 also includes input/output 640, representing devices which allow for user interaction with a computer (e.g., display, keyboard, mouse, speakers, buttons, etc.). One skilled in the art will recognize that an implementation of an actual device will contain other components as well, and that FIG. 4 is a high level representation of some of the components of such a device, for illustrative purposes. It should also be understood by one skilled in the art that the method and devices depicted in FIGS. 1 through 3 may be implemented on a device such as is shown in FIG. 4.

Further, it should be understood that all subject matter disclosed herein is directed, and should be read, only on statutory, non-abstract subject matter. All terminology should be read to include only the portions of the definitions which may be claimed. By way of example, “computer readable storage medium” is understood to be defined as only non-transitory storage media.

While the disclosed technology has been taught with specific reference to the above embodiments, a person having ordinary skill in the art will recognize that changes can be made in form and detail without departing from the spirit and the scope of the disclosed technology. The described embodiments are to be considered in all respects only as illustrative and not restrictive. All changes that come within the meaning and range of equivalency of the claims are to be embraced within their scope. Combinations of any of the methods, systems, and devices described herein-above are also contemplated and within the scope of the disclosed technology.

I claim:

1. A method of short range communication, comprising the steps of:
distributing a first and second short range communication device with a data storage device, respectively, to a first and a second health care practitioner;
associating a unique identifier stored in each said data storage device with each respective said first and said second health care practitioner;
associating a first plurality of patients with a first health care practitioner of said two health care practitioners;
receiving an indication that a particular patient of said plurality of patients is no longer under care of said first health care practitioner and removing said associating of said particular patient from said first health care practitioner;
receiving via a network node an indication of a wireless electric transmission of data between said short range communication device of said first health care practitioner with said short range communication device of said second health care practitioner;
associating said plurality of patients with said second health care practitioner.

2. The method of claim 1, further comprising a step of:
sending information about each of said first plurality of patients to a handheld wireless transceiver associated with said first health care practitioner after said step of associating said first plurality of patients with said first health care practitioner.

3. The method of claim 2, further comprising a step of:
sending information about each of said first plurality of patients to a handheld wireless transceiver associated with said second health care practitioner after said step of receiving said indication of said wireless electric transmission of data.

4. The method of claim 3, wherein patient information about a patient of said first plurality of patients, including either patient care data or patient vital statistics, is received by way of said handheld wireless transceiver associated with said first health care practitioner.

5. The method of claim 4, wherein said patient care data or said patient vital statistics are unavailable to said second health care practitioner until after said step of receiving said indication of said wireless electric transmission of data.

6. The method of claim 1, wherein said first and second short range communication devices comprise wrist bands.

7. The method of claim 6, wherein said first and second short range communication devices communicate directly with each other and to said network node.

8. The method of claim 7, wherein data from said network node is made available to at least a separate device with network connectivity confirmed to be operated by a third health care professional.

9. A system, comprising the steps of:
distributing short range communication devices to a plurality of health care professionals;
based on an unique identifier within each of said short range communication devices, associating each said short range communication device thereof with one of a plurality of health care professionals;
receiving via a network node an indication of at least one patient under care of a first health care professional of said plurality of health care professionals;
receiving an indication that a direct data exchange has taken place between two of said short range communication devices, wherein a first of said short range communication devices is associated with said first health care professional and a second of said short range communication devices is associated with a second of said health care professionals;
via a network node, receiving a request for care of said at least one patient and, based on said direct data exchange, sending said second health care professional for said care.

10. The system of claim 9, wherein upon detecting that said first health care professional has exited from premises, sending a reminder via a packet-switched network node to a handheld device previously determined to be operated by said first health care professional to transfer said at least one patient to another of said health care professionals.

11. The system of claim 9, wherein upon determining that a work shift is over for said first health care professional, sending a reminder via a packet-switched network node to a handheld device previously determined to be operated by said first health care professional to transfer said at least one patient to another of said health care professionals.

12. The system of claim 9, wherein said short range communication devices communicate patient identifying information to one another whom are under care of said first health care professional, and after communication of said patient identifying information, at least a device previously determined to be operated by said second health care professional is sent a notification that said second health care provider is responsible for said at least one patient.

13. The system of claim 12, wherein a query is received via a network node requesting contact information for a responsible health care professional for said at least one patient and a response is returned indicating a latest recipient of said direct data exchange between two said short range communication devices.

14. The system of claim 12, wherein at least one of said short range communication devices communicates patient identifying information to a device providing data sufficient to answer said query.

15. The system of claim 9, wherein said short range communication devices communicate substantially only said unique identifier to each other, and substantially only said unique identifier is transmitted wirelessly to a data storage device which stores an association between said at least one patient and, at any given time, one of said plurality of health care professionals.

16. The system of claim 15, further comprising a fixed position reader with a transceiver communicating with said short range communication devices, wherein said step of receiving an indication that said direct data exchange has taken place further comprises the steps of:
said fixed position reader receives data from said short range communication device associated with said first health care professional sufficient to indicate that said at least one patient associated with said first health care professional is now associated with said second health care professional;
said first position reader receives data from said short range communication device associated with said second health care professional sufficient to indicate that said at least one patient associated with said first health care professional is now associated with said second health care professional.

17. A method of patient care, comprising the steps of:
carrying an identification device with wireless connectivity;
locating or being located by another health care professional whose duties are to be assumed;
placing said identification device close enough and for a long enough time period until a wireless data exchange takes place between said identification device and that of a second identification device associated with said professional whose duties are to be assumed;
causing information about said wireless data exchange to be transmitted via a network node to a server;
receiving from said server an indication of patients of said another health care professional for which duties are assumed.

18. The method of claim 17, further comprising a step of receiving a reminder, exhibited by said identification device, to carry out said steps of locating and placing.

19. The method of claim 18, wherein said reminder is triggered by said identification device accelerating about a pre-defined rate of acceleration or traveling at a velocity above a pre-defined rate of velocity.

20. The method of claim 19, wherein said reminder is triggered by said identification device exiting from a geo-fenced location.
